SEQUENCE LISTING (8 sequences)

SEQ ID NO: 1 - DNA, 21 bp, artificial sequence
  Synthetic oligonucleotide
cgcattcctcatcccagtatg

SEQ ID NO: 2 - DNA, 23 bp, Artificial sequence
  Synthetic oligonucleotide
aaaggacttggtgcagagttcag

SEQ ID NO: 3 - DNA, 17 bp, Artificial sequence
  Synthetic oligonucleotide
cccaccgccttcgacat

SEQ ID NO: 4 - DNA, 21 bp, Artifical sequence
  Synthetic oligonucleotide
cctgcttactgtgggctcttg

SEQ ID NO: 5 - DNA, 26 bp, Artificial sequence
  Synthetic oligonucleotide
ctcgcttaaccagactcatctactgt

SEQ ID NO: 6 - DNA, 23 bp, Artificial sequence
  Synthetic oligonucleotide
acttggctcagctgtatgaaggt

SEQ ID NO: 7 - DNA, 20 bp, Artificial sequence
  Synthetic oligonucleotide
cagccccagctcaggtacag

SEQ ID NO: 8 - DNA, 21 bp, Artificial sequence
  Synthetic oligonucleotide
atgatgcgagtgctgatgatg

(19) United States
(12) Patent Application Publication
(10) Pub. No.: US 2009/0203540 A1
(43) Pub. Date: Aug. 13, 2009

(54) Methods and Systems for Quality Control Metrics in Hybridization Assays

(75) Inventors: CHRISTINA MIDDLE, MADISON, WI (US); JACOB KITZMAN, MADISON, WI (US); TODD RICHMOND, MADISON, WI (US); THOMAS ALBERT, MADISON, WI (US); JEFFREY JEDDELOH, VERONA, WI (US)

Correspondence Address: Roche NimbleGen, 500 S. Rosa Road, Madison, WI, 53719, US

(73) Assignee: ROCHE NIMBLEGEN, INC., MADISON, WI (US)

(21) Appl. No.: 12/363,897

(22) Filed: Feb. 2, 2009

Related U.S. Application Data
(60) Provisional application No. 61/026,596, filed on Feb. 6, 2008.

(51) Int. Cl. C40B 30/04; C12Q 1/68; C40B 40/06

(52) U.S. Cl. 506/9; 435/6; 506/16

(57) ABSTRACT

The present invention provides methods and systems for performing quality control metrics in hybridization assays. In particular, the present invention provides for quality control metrics for nucleic acid enrichment on hybridization assay formats, such as microarray assays.

This patent application claims priority from the U.S. Provisional Patent Application Ser. No. 61/026,596, filed Feb. 6, 2008, incorporated herein by reference in its entirety.

### FIELD OF THE INVENTION

The present invention provides methods and systems for performing quality control metrics in hybridization assays. In particular, the present invention provides for quality control metrics for nucleic acid enrichment on hybridization assay formats, such as microarray assays.

### BACKGROUND OF THE INVENTION

The advent of nucleic acid microarray technology makes it possible to build an array of millions of nucleic acid sequences in a very small area, for example on a microscope slide (e.g., U.S. Pat. Nos. 6,375,903 and 5,143,854). Initially, such arrays were created by spotting pre-synthesized DNA sequences onto slides. However, the construction of maskless array synthesizers (MAS) as described in U.S. Pat. No. 6,375,903 now allows for the in situ synthesis of oligonucleotide sequences directly on the slide itself.

Using a MAS instrument, the selection of oligonucleotide sequences to be constructed on the microarray is under software control such that it is now possible to create individually customized arrays based on the particular needs of an investigator. In general, MAS-based oligonucleotide microarray synthesis technology allows for the parallel synthesis of over 4 million unique oligonucleotide features in a very small area of a standard microscope slide. With the availability of the entire genomes of hundreds of organisms, for which a reference sequence has generally been deposited into a public database, microarrays have been used to perform sequence analysis on nucleic acids isolated from a myriad of organisms.

Nucleic acid microarray technology has been applied to many areas of research and diagnostics, such as gene expression and discovery, mutation detection, allelic and evolutionary sequence comparison, genome mapping, drug discovery, and more. Many applications require searching for genetic variants and mutations across the entire human genome; variants and mutations that, for example, may underlie human diseases. In the case of complex diseases, these searches generally result in a single nucleotide polymorphism (SNP) or set of SNPs associated with one or more diseases. Identifying such SNPs has proven to be an arduous, time consuming, and costly task wherein resequencing large regions of genomic DNA, usually greater than 100 kilobases (Kb) from affected individuals and/or tissue samples is frequently required to find a single base change or identify all sequence variants.

The genome is typically too complex to be studied as a whole, and techniques must be used to reduce the complexity of the genome. To address this problem, one solution is to reduce certain types of abundant sequences from a DNA sample, as found in U.S. Pat. No. 6,013,440. Other alternatives employ methods and compositions for enriching genomic sequences as described, for example, in Albert et al. (2007, Nat. Meth., 4:903-5). Albert et al. disclose an alternative that is both cost-effective and rapid in effectively reducing the complexity of a genomic sample in a user defined way to allow for further processing and analysis.

However, it is equally important to be able to identify the amount and/or extent of enrichment that has occurred from practicing, for example, the methods as described by Albert et al., or other methods that utilize nucleic acid enrichment with microarray formats. As such, what are needed are methods and compositions to complement such microarray technologies thereby providing maximum data utility to investigators in their endeavors to understand and identify, for example, causes of disease and associated therapeutic treatments.

### SUMMARY OF THE INVENTION

The present invention provides methods and systems for performing quality control metrics in hybridization assays. In particular, the present invention provides for quality control metrics for nucleic acid enrichment on hybridization assay formats, such as microarray assays.

Nucleic acid enrichment reduces the complexity of a large nucleic acid sample, such as a genomic DNA sample, cDNA library or mRNA library, to facilitate further processing and genetic analysis. Pre-existing nucleic acid capture methods utilize immobilized nucleic acid probes to capture target nucleic acid sequences (e.g. as found in genomic DNA, cDNA, mRNA, etc.) by hybridizing the sample to probes immobilized on a solid support. The captured target nucleic acids, for example found in genomic DNA, are preferably washed and eluted off of the solid support-immobilized probes. The eluted genomic sequences are more amenable to detailed genetic analysis than a genomic sample that has not been subjected to this procedure. Enrichment of target nucleic acid sequences takes nucleic acid capture one step further, by reducing the complexity of a sample wherein sequences of interest are selected for, or enriched, by selective processes. Enrichment methods and compositions are fully disclosed in U.S. patent application Ser. No. 11/789,135 and 11/970, 949 and World Intellectual Property Organization Application Number PCT/US07/010064, all of which of incorporated herein by reference in their entireties.

As described herein and in prior applications as previously listed, enrichment of target nucleic acids in a microarray format is important in reducing the complexity of a nucleic acid sample prior to, for example, sequencing or other downstream applications. Of equal import is the need for methods and systems to evaluate and access the enrichment methods and to evaluate the quality of nucleic acids recovered from such methods. As such, described herein are methods and materials for accessing the quality of nucleic acids post-enrichment in a hybridization format, such as a microarray.

Certain illustrative embodiments of the invention are described below. The present invention is not limited to these embodiments.

In some embodiments, the present invention comprises a solid support microarray, generally comprising (pre-selected) support-immobilized nucleic acid probes to capture and enrich for specific nucleic acid sequences (target nucleic acids) from a sample (e.g., genomic DNA, cDNA, mRNA, tRNA, etc.). In some embodiments, target nucleic acid enrichment is via hybridizing a nucleic acid sample, for example a genomic DNA sample, which may contain one or more target nucleic acid sequence(s), against a microarray having array-immobilized nucleic acid probes directed to a specific region or specific regions of the genome. After hybridization, target nucleic acid sequences present in the sample are enriched by washing the array and eluting the hybridized genomic nucleic acids from the array. Following elution, the enriched samples are assayed for the level, or amount of enrichment over a control, and the fold enrichment is calculated thereby determining the quality of the enriched sample. In some embodiments, the target nucleic acid sequence(s) are further amplified using, for example, non-specific ligation-mediated PCR (LM-PCR), resulting in an amplified pool of PCR products of reduced complexity compared to the original (genomic) sample for sequencing, library construction, and other applications.

In some embodiments, the present invention comprises a solid support microarray, generally comprising (pre-selected) support-immobilized nucleic acid probes to capture specific nucleic acid sequences (target nucleic acids) from a sample (e.g., genomic DNA, cDNA, mRNA, tRNA, etc.). In some embodiments, the sample is fragmented, for example by sonication, or other methods capable of fragmenting nucleic acids. In some embodiments, the fragmented sample (e.g., fragmented genomic DNA, cDNA, etc.) is modified by ligation to linkers on one or both of the 5′ and 3′ ends. In some embodiments, the 5′ and 3′ ends of a fragmented sample are first prepared for ligation with a linker, for example by performing a “fill in” reaction with Klenow enzyme. The preparation of nucleic acid ends for subsequent ligation to linkers is well known in the art, and can be found in any molecular cloning manual such as “Molecular Cloning: A Laboratory Manual, Sambrook et al. Eds, Cold Spring Harbor Laboratory Press”, which is herein incorporated be reference in its entirety. Indeed, all molecular cloning, hybridization, washing, and elution techniques as used herein can be found in Molecular Cloning: A Laboratory Manual as well as “A Molecular Cloning Manual: DNA Microarrays”, Bowtell et al., Eds, Cold Spring Harbor Press (incorporated herein by reference in its entirety). In some embodiments, the fragmented and linker-modified nucleic acid sample is hybridized to an array comprising probes designed to capture target sequences, and the targeted sequences are captured. The use of linkers for enrichment methods and enrichment methods in general are well known and fully described in U.S. patent application Ser. No. 11/789,135 and 11/970,949 and World Intellectual Property Organization Application Number PCT/US07/010064, and further in Albert et al. (2007) and Okou et al. (2007); all of which of incorporated herein by reference in their entireties.

Following hybridization, non-targeted nucleic acids are washed from the microarray and the bound, targeted nucleic acids are eluted from the microarray. The quality of the enriched sample is calculated and fold enrichment is determined and communicated to the user. In some embodiments, the calculation of enrichment comprises fold enrichment as compared in a control enrichment sample. Samples of sufficient quality (e.g., enrichment) are used for downstream applications, such as sequencing, cloning, library construction, etc.

The present invention is not limited by any downstream use of enriched nucleic acids, and a skilled artisan will understand the myriad uses such a sample would provide, such as SNP detection for discovery and correlation with disease states and risk factors, use of targeted sequences in drug discovery applications, etc.

The present invention provides for the assessing of the quality of microarray based enriched target nucleic acids (e.g., level of effectiveness of the enrichment methods) as described herein. The assessment not only provides insight into the general effectiveness of the enrichment technology, but it also provides an investigator a method of accessing the quality of the enriched nucleic acids prior to spending precious time and resources on downstream applications with a sample that is not appropriately enriched. In some embodiments, the assessing of the quality of the target nucleic acids is performed by testing the enrichment of a subset of reference sequences, for example conserved regions in a genome (FIG. 1). The present invention is not limited to the location of the conserved regions, and any conserved regions are contemplated as useful in methods for assessing the quality of enrichment as described herein.

In some embodiments, once conserved regions in a sequence are identified for evaluation, primers are designed against locations in the conserved regions such that, for example, quantitative PCR (qPCR) measurements are performed on the conserved regions pre and post enrichment (e.g., hybridization and washing, etc.). Using such measuring techniques, levels of sample enrichment pre and post enrichment are determined. However, the present invention is not limited to the measuring techniques used to determine the levels of sample enrichment as defined by enrichment of conserved regions, and any method for comparison evaluation of conserved regions is contemplated (e.g., PCR, Northern blot analysis, radioactive labeling assays, fluorescent tags, antibody binding assays, etc.). In developing embodiments of the present invention, the quality control methods as described herein for determining sample quality enrichment were further validated by subsequent sequencing of the enriched sample thereby correlating the effectiveness of enrichment of the reference sequences with the overall effectiveness of the enriched target sequences. Indeed, the high correlation demonstrated during experimentation between the effectiveness of enrichment of the reference sequences and the enrichment of the target sequences validates the methods as described herein for evaluating quality of microarray based sequence enrichment methods.

In one embodiment, the present invention provides methods for determining enrichment of nucleic acid sequences from a hybridization assay comprising providing a nucleic acid sample comprising conserved nucleic acid sequences, probes comprising the conserved nucleic acid sequences as found on the nucleic acid sample, hybridizing the nucleic acid sample with the probes thereby capturing the conserved nucleic acid sequences, and comparing the amount of conserved nucleic acid sequences captured before hybridization to the amount of conserved nucleic acid sequences captured after hybridization, thereby determining enrichment of nucleic acid sequences. In some embodiments, the nucleic acid sequences further comprise target nucleic acid sequences. In some embodiments, the hybridization assay is a microarray assay. In some embodiments, the nucleic acid sequences are genomic DNA sequences. In some embodiments, comparing comprises performing polymerase chain reaction on the captured conserved nucleic acid sequences before hybridization and after hybridization. In some embodiments, the polymerase chain reaction is preferably quantitative PCR. In some embodiments, determining the enrichment comprises determining fold enrichment between the amount of nucleic acid sequences captures prior to hybridization as compared to after hybridization. In some embodiments, the enriching comprising removing non-hybridized nucleic acids by washing, further by eluting the hybridized and washed nucleic acid sequences.

In one embodiment, the present invention provides methods for determining enrichment of nucleic acid sequences from a microarray assay comprising providing a nucleic acid sample comprising conserved and target nucleic acid sequences, applying said sample to a substrate wherein said substrate comprises probes hybridizable to said conserved nucleic acid sequences, allowing hybridization capture to occur between the same and said probes, washing and eluting the captured nucleic acid sequences and comparing the amount of conserved nucleic acid sequences captured before hybridization to the amount of conserved nucleic acid sequences captured after hybridization, thereby determining enrichment of nucleic acid sequences from a microarray assay.

In one embodiment, the present invention provides kits for determining enrichment of nucleic acid sequences in a hybridization assay comprising a substrate, probes affixed to said substrate wherein said probes are homologous to one or more conserved nucleic acid sequences in a sample and primers homologous to said conserved nucleic acid sequences, wherein said primers are capable of performing polymerase chain reactions of said conserved nucleic acid sequences for determining enrichment of nucleic acid sequences. In some embodiments, the kits further comprise reagents or solutions for performing hybridizations, washings, and elusions. In some embodiments, kits further comprise one or more of a polymerase, a ligase, a kinase and a terminal transferase.

### Definitions

As used here, the term “substrate” is used in reference to a surface in its broadest sense. Substrates of the present invention comprise glass or plastic slides, chips, or other linear surface. Substrates also include tubes, beads, rods, be they made of glass, plastic, or any other composition. In the context of microarrays, substrates further comprise immobilized probes, such that probe sequences designed to be hybridizable to nucleic acid and/or peptide sequences are either synthesized directly (e.g., in situ synthesis such as MAS) or applied (e.g., spotted onto, etc.) onto a substrate.

As used herein, the term “hybridization” and “hybridizable” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement (e.g. probe sequence), sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. In the context of the present invention, a probe is typically immobilized on a substrate and is designed to capture (e.g., hybridize to) a target sequence, such as a nucleic acid or peptide sequence, resulting in the enriching of that target sequence over other nucleic acid sequences found in a sample.

As used herein, the term “sample” is used in its broadest sense. In one sense, it is meant to include a specimen or culture obtained from any source, as well as biological and environmental samples. In the context of the present invention, a sample is preferably a nucleic acid or peptide sample. A nucleic acid sample of the present invention can be DNA, RNA, genomic, fragmented, and the like. Biological nucleic acid and/or peptide samples may be obtained from animals (including humans) and encompass fluids, solids, tissues, and gases. Such examples are not however to be construed as limiting the sample types applicable to the present invention. Samples include nucleic acid sequences that comprise target sequences capable of hybridizing to complementary (e.g., either partially or wholly complementary) probe sequences. Target nucleic acid sequences comprise sequences of interest to investigators. A sample further comprises sequences that are considered conserved intra and inter species genomic conserved sequences, for example such that a certain sequence is homologous between humans, non-human primates (as well within primate species), rodents (as well within rodent species), and the like.

The term “hybridization assay” as used herein, refers to any type of assay where hybridization is used. Examples herein are microarray assays, however the quality controls metrics as defined herein are equally amenable to any hybridization assay, such as nucleic acid blots (e.g., northern, southern) and the like. As well, protein hybridization assays (e.g., western blots, etc.) are also applicable if the conserved regions used are amino acid sequences in lieu of nucleic acid sequences.

### DESCRIPTION OF THE FIGURES

FIG. 1 demonstrates exemplary conserved regions as compared between nondescript human and mouse genomic DNA.

FIG. 2 demonstrates one embodiment of quality controlling for input enriched DNA prior to sequencing.

FIG. 3 shows an exemplary fold enrichment of target genomic sequences as determined by quantitative PCR quality control methods.

FIG. 4 demonstrates the repeatability of the enrichment technology as determined by quantitative PCR.

FIG. 5 demonstrates an exemplary use of quality control in identifying samples that have been enriched; A) a sample with highly enriched target sequences, B) a sample with questionable enrichment (potential DNA degradation), and C) an enriched compromised, bisulfite converted sample.

FIG. 6 demonstrates an exemplary increase in effective concentration mass unit of target sequences pre and post enrichment.

FIG. 7 shows the averages of four control loci (Table 1) from each of two enrichment experiments for each design. The first seven results (left to right) denote human genomic enrichment experiments, whereas the last three are results from mouse genomic sequence enrichment experiments.

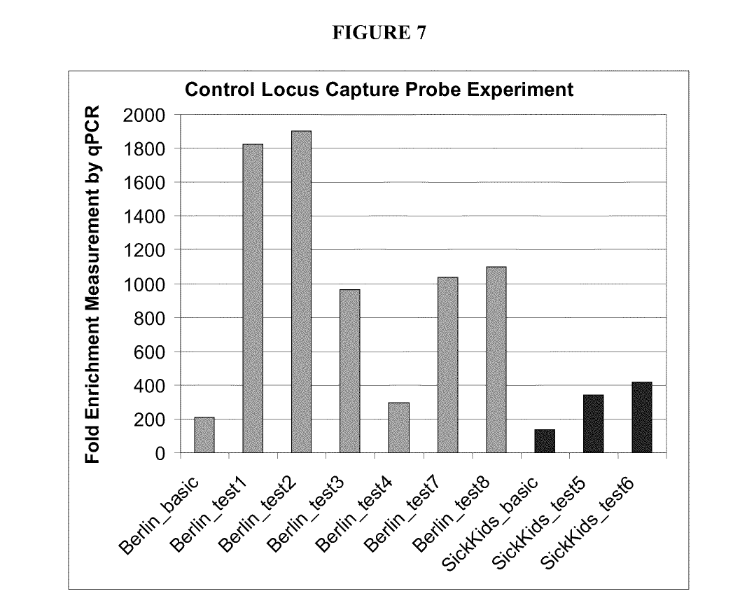

### DETAILED DESCRIPTION OF THE INVENTION

Targeted genomic sequencing is one of the most important biomedical applications of next-generation sequencing technologies. A revolutionary way to target next generation sequencing utilizes oligonucleotide microarrays as sample preparation devices. These arrays capture regions of the genome defined by the array probes, which are then eluted and, for example, sequenced. Because of the relatively high per run cost of next generation sequencing, it is important to have robust quality control metrics that ensure that only samples that are highly enriched for target regions are sequenced. Two important characteristics of successfully captured samples are 1) highly enriched for targeted regions, and 2) uniformly enriched across all targeted regions. To this end, the present invention comprises assays that are highly predictive of subsequent sequencing data quality for captured nucleic acids, for example genomic DNA. It is contemplated that a key aspect in the quality control process are assays that query for a set of control regions targeted for capture, including for example highly conserved regions in loci across all mammals. In developing embodiments of the present invention, it was discovered that quantitative PCR assays provide a rapid, high-throughput, and low cost measurement of enrichment performance. As such, described herein are methods and materials demonstrating quality control metrics that predict fold enrichment levels of samples consistent with sequencing results.

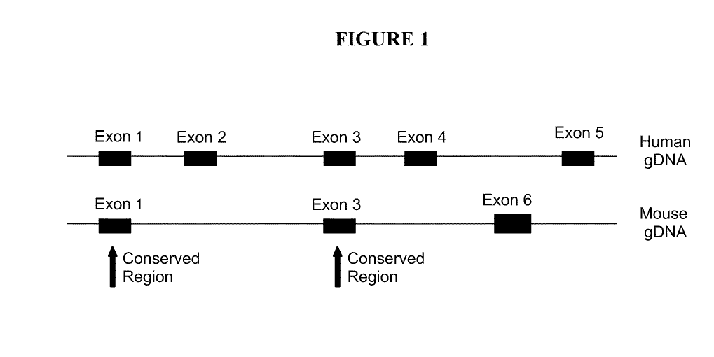

In developing embodiments of the present invention, it was determined that comparisons for evaluating enrichment methods were most efficacious when conserved regions across mammals were chosen for comparison (FIG. 1). In some embodiments, the conserved regions chosen for comparison are found in mammals. Conserved regions in genomes are identified in a myriad of ways, for example by sequence alignment. Sequences for alignment are found in many depositories, and are typically open to public use (e.g., NCBI GenBank and other public databases). As well, there are multiple programs available for aligning sequences, such as BLAST, and tools found at EMBL (European Bioinformatics Institute hosted website). However, the present invention is not limited by the method used for determining conserved regions, and any method is amenable for use with the present invention. It is contemplated that conserved regions useful in evaluating enrichment methods are not limited to those of mammals, and conserved sequences for comparisons between non-mammalian genomes are also contemplated, for example depending on the source of the sample to be enriched. In some embodiments, pan-mammalian loci are utilized in evaluating enrichment methods for quality sample determinations, thereby decreasing the necessity to design enrichment-locus specific controls (e.g., conserved regions) for human, primate, and rodent experiments. Indeed, it is contemplated that non-conserved regions are also amenable to use in quality control metrics of the present invention, in so far that such regions serve as sites for comparison and evaluation for sample enrichment.

In some embodiments, the quality control method of choice is preferably quantitative PCR, and once conserved regions for comparison are defined primer pairs are designed for performing the quantitative PCR on conserved region sequences. However, it is contemplated that other methods for quantitating conserved regions are applicable for use in methods of the present invention, and a skilled artisan will appreciate alternative methods of quantitating the conserved regions as defined herein.

The following examples are provided in order to demonstrate and further illustrate certain preferred embodiments and aspects of the present invention and are not to be construed as limiting the scope thereof.

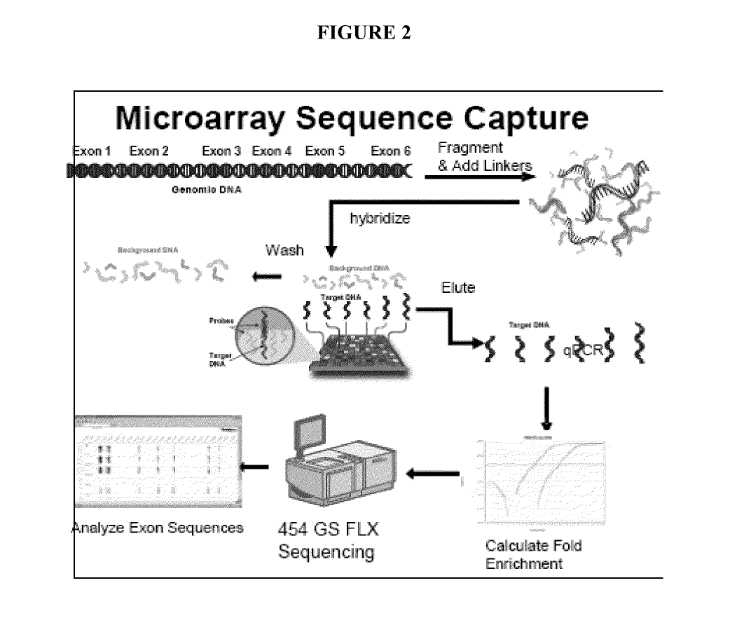

FIG. 2 depicts an exemplary enrichment experiment incorporating an embodiment of a quality assessment method of the present invention. For example, a genomic DNA sample which may contain one or more target sequences is fragmented and modified by incorporation of linkers and subsequently hybridized to probes as found on a microarray, wherein said probes are specific to one or more target regions on a genomic DNA sample. Some of the sample is maintained for quality testing and not applied to the microarray (PRE microarray sample). In developing embodiments of the present invention, samples were exposed to a variety of insults, for example some samples were “compromised” by exposure to bisulfite, whereas other samples were deemed of “questionable” quality, for example some samples were degraded. The samples were applied to microarrays for enrichment, hybridizations were performed, and unbound sample was washed from the microarray. Subsequent bound target sequences were eluted, a sample of which was maintained for quality testing (POST microarray sample). In some embodiments, the PRE and POST samples were amplified and the amplicons quantitated using quantitative PCR (qPCR). Equal mass of each amplification reaction was determined and compared for a change in concentration pre and post enrichment. For example, the change in concentration is calculated such that:

ΔCt=ratio(PRE/POST) in linear space and or ΔCt(PRE/POST)log space

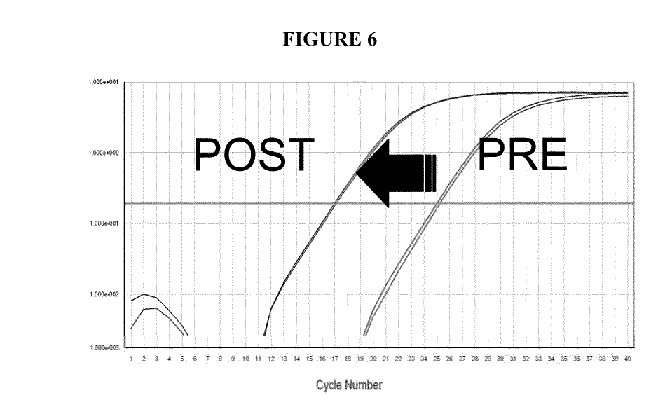

It is contemplated that as the array enriches for copies of the target region at the expense of other regions (e.g., non-targeted regions), there is an increase in the effective concentration per unit of mass (FIG. 6). As such, one embodiment of the present invention is the evaluation of sample quality enrichment by comparison of concentration changes between the chosen conserved regions in a sample pre and post enrichment.

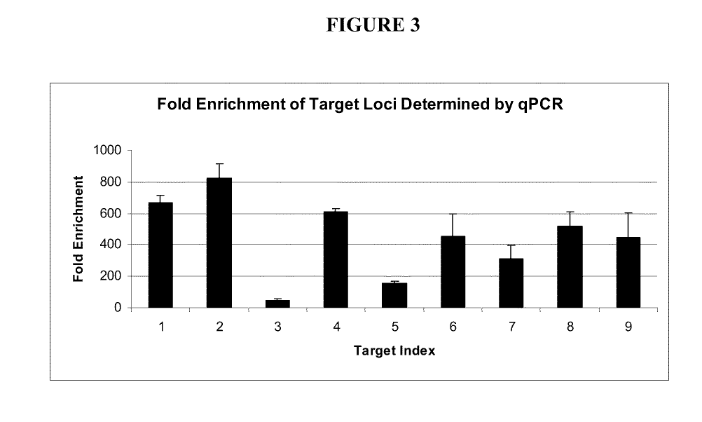

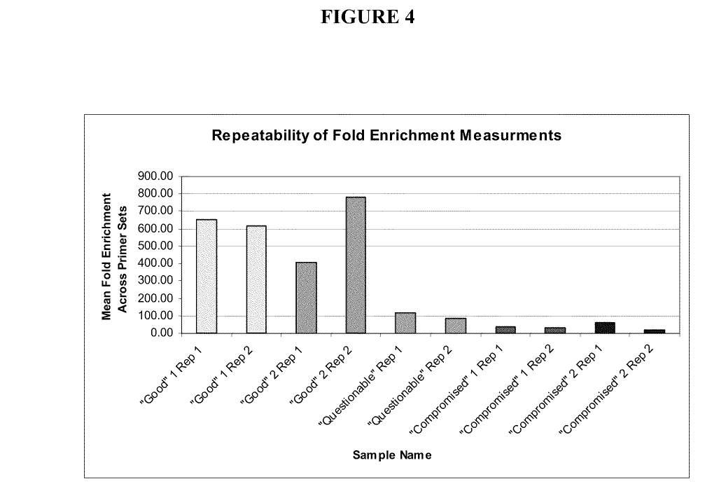

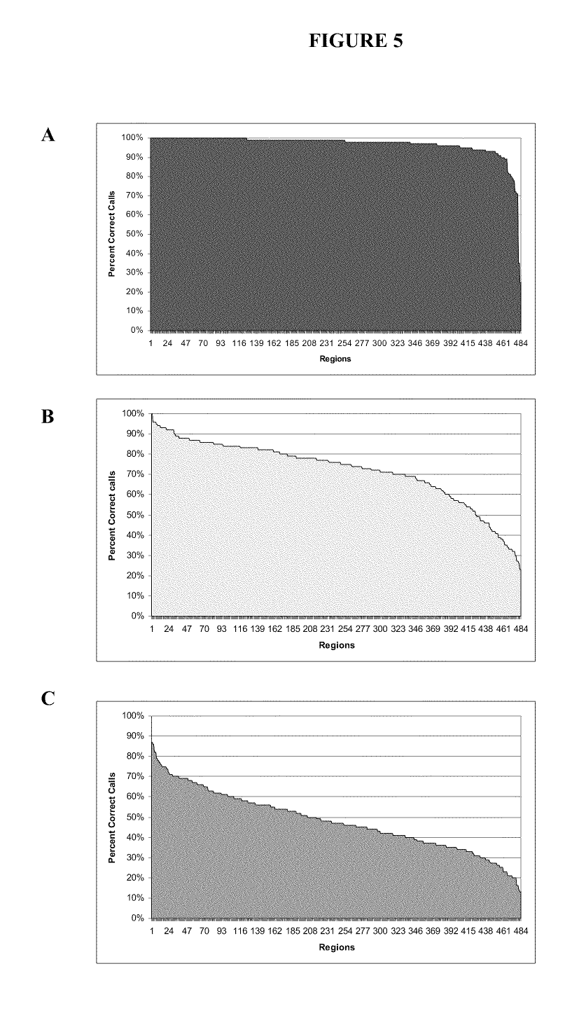

Microarray resequencing requires genome complexity reduction to interrogate specific loci. This resequencing is typically performed by amplicon sequencing; however the present invention is not limited to the sequences used for resequencing, as non-amplified nucleic acids are also contemplated for use as resequencing templates. Enrichment methods as described herein and as found in U.S. patent application Ser. No. 11/789,135 and 11/970,949 and World Intellectual Property Organization Application Number PCT/US07/010064 and as described in Albert et al. (2007) and Okou et al. (2007, Nat. Meth. 11:907-909; incorporated herein by reference in its entirety) are used to prepare target loci for microarray sequencing. If enrichment is successful, such that the target sequences were indeed selected for on the enrichment array, resequencing arrays will demonstrate a high mean conformance (e.g., at least 75%, at least 80%, at least 85%, at least 90%, at least 95%, at least 97%, at least 99%). Examples of differentially enriched samples as determined by the quality control metrics described herein are seen in FIG. 5. FIG. 5 demonstrates the resequencing success, depicted as percent of correct calls on resequence, of several different sample types. As can be seen in FIGS. 5A-C, as the calculated mean fold enrichment (as determined using qPCR data) increased (A-mean fold enrichment (qPCR) 632.96; B-100.69; C-71.7) so did the percentage of correct calls on resequence. Mean resequencing conformance for FIGS. 5A-3 were 97%, 72% and 48%, respectively. FIG. 4 demonstrates the repeatability of the fold enrichment measurements of the present invention, thereby further demonstrating the efficacy of utilizing methods of the present invention in determining quality enrichment samples.

Pre-existing experimental data was also applied to the quality control metrics as described herein (Albert et al., 2007). Briefly, 6726 loci were enriched using microarray enrichment. These loci represent approximately 5 Mb of total sequence, or approximately 0.15% of the genome. Nine conserved regions of loci were chosen for pPCR, and average fold enrichment was determined by qPCR to be 428 fold enrichment. Samples were sequenced using 454 GSFLX sequencing instrument (454, Branford, Conn.) and fold enrichment was determined to be 432 (FIG. 3). As such, quality assessment of target enrichment utilizing methods of the present invention, wherein conserved regions are used for comparison evaluation as described herein, are useful in predicting sample enrichment quality prior to resequencing or other downstream applications. Such predictions and determinations provide a useful tool to investigators in evaluating enriched samples prior to spending money and resources on potentially problematic samples.

As such, methods and materials of the present invention for evaluating the quality of target sequences enriched using enrichment microarray technologies provides rapid, low cost methods for determining microarray enrichment success. For example, the quantification of control loci fold enrichment eliminates costly sequencing of poorly enriched samples thereby saving time and money. The small numbers of probes used to capture the conserved quality control regions can be easily incorporated into a microarray format without detracting from target sequence capture, as such the quality control materials are easily insertable into a microarray for concurrent capture with an investigator's target sequences. Further, it is contemplated that the incorporation of pan-mammalian loci can serve as cross species conserved regions, thereby alleviating the need for separate controls for human, primate, and rodent experiments.

In some embodiments, the present invention provides kits for practicing methods and assays as described herein. In some embodiments, the kits comprise reagents and/or other components (e.g., buffers, instructions, solid surfaces, containers, software, etc.) sufficient for, necessary for, performing target nucleic acid capture of target nucleic acid molecules and conserved nucleic acid molecules as herein described. Kits are provided to a user in one or more containers (further comprising one or more tubes, packages, etc.) that may require differential storage, for example differential storage of kit components/reagents due to light, temperature, etc. requirements particular to each kit component/reagent. In some embodiments, a kit comprises one or more solid supports, wherein said solid support is a microarray slide or a plurality of beads, upon which are affixed a plurality of oligonucleotide capture probes. In some embodiments, a kit comprises oligonucleotide probes in solution, wherein said probes comprise a capture moiety, and beads, wherein said beads are designed to bind to the capture moiety as affixed to the oligonucleotide probe. For example, such a moiety is a biotin label which can be used for immobilization on a streptavidin coated solid support. Alternatively, such a modification is a hapten like digoxygenin, which can be used for immobilization on a solid support coated with a hapten recognizing antibody.

In some embodiments, the present invention comprises kits comprising at least one or more compounds and reagents for performing enzymatic reactions, for example one or more of a thermostable DNA polymerase, a T4 polynucleotide kinase, a restriction endonuclease, a terminal transferase, Klenow, etc. In some embodiments, a kit comprises one or more of hybridization solutions, wash solutions, and/or elution reagents. Examples of wash solutions found in a kit include, but are not limited to, Wash Buffer I (0.2×SSC, 0.2% (v/v) SDS, 0.1 mM DTT), and/or Wash Buffer II (0.2×SSC, 0.1 mM DTT) and/or Wash Buffer III (0.5×SSC, 0.1 mM DTT). In some embodiments, a kit comprises one or more elution solutions, wherein said elution solutions comprise purified water and/or a solution containing TRIS buffer and/or EDTA, or other low solute solution.

The following examples are provided in order to demonstrate and further illustrate certain embodiments and aspects of the present invention and are not to be construed as limiting the scope thereof.

### EXAMPLE 1

### Use of qPCR and Control Capture loci to Assess Sequence Capture Enrichment

The following exemplary experiments demonstrate methods of using qPCR and control capture loci to assess enrichment of targeted and captured sequences in human and non-human genomes, methods which can be applied to assess enrichment in any species regardless of origin. Through experimentation it was determined that several factors resulted in a significant increase in the enrichment of the control capture loci as measured by qPCR relative quantification. For example, it was determined that 1) an increase in the density of capture probes targeting the control loci, (2) an increase in the copy-number of control locus capture probes on the oligonucleotide array, and (3) use of control locus capture probes whose nucleotide sequence precisely matches (i.e. “isogenic”) the species targeted for enrichment resulted in an increase in control loci enrichment.

Sequence capture (e.g., enrichment) arrays were manufactured by Roche NimbleGen, Inc. using maskless array synthesis as previously identified. Ten different designs were used for creating control loci enrichment arrays, the genetic sequences of which were obtained from the University of California Santa Cruz Genome Bioinformatics database (UCSC Genome Bioinformatics Site http://genome.UCSC.edu/). Probes on arrays were designed, seven of which were designed to demonstrate human genomic sequence enrichment (HG18) and three were designed to demonstrate murine genomic sequence enrichment (MM9).
1. “Berlin_basic” is identical to design “080905_HG18_Berlin_{—}9q31_{—}32_cap”. This design targets 188,633 bp of the human genome for capture.
2. “Berlin_test1” is identical to design “080905_HG18_Berlin_{—}9q31_{—}32_cap” except that the capture probes for the control loci have been altered as described in Table 1. The full design name is: “080918_HG18_QC_test1_cap”.
3. “Berlin_test2” is identical to design “080905_HG18_Berlin_{—}9q31_{—}32_cap” except that the capture probes for the control loci have been altered as described in Table 1. The full design name is: “080918_HG18_QC_test2_cap”.
4. “Berlin_test3” is identical to design “080905_HG18_Berlin_{—}9q31_{—}32_cap” except that the capture probes for the control loci have been altered as described in Table 1. The full design name is: “080918_HG18_QC_test3_cap”.
5. “Berlin_test4” is identical to design “080905_HG18_Berlin_{—}9q31_{—}32_cap” except that the capture probes for the control loci have been altered as described in Table 1. The full design name is: “080918_HG18_QC_test4_cap”.
6. “Berlin_test7” is identical to design “080905_HG18_Berlin_{—}9q31_{—}32_cap” except that the capture probes for the control loci have been altered as described in Table 1. The full design name is: “080918_HG18_QC_test7_cap”.
7. “Berlin_test8” is identical to design “080905_HG18_Berlin_{—}9q31_{—}32_cap” except that the capture probes for the control loci have been altered as described in Table 1. The full design name is: “080918_HG18_QC_test8_cap”.
8. “SickKids_basic” is identical to design “080701_MM9_SickKids_JD_cap”. This design targets 1,089,653 bp of the mouse genome for capture.
9. “SickKids_test5” is identical to design “080701_MM₉_SickKids_JD_cap” except that the capture probes for the control loci have been altered as described in Table 1. The full design name is: “080918_MM9_QC_test5_cap”.
10. “SickKids_test6” is identical to design “080701_MM9_SickKids_JD_cap” except that the capture probes for the control loci have been altered as described in Table 1. The full design name is: “080918_MM9_QC_test6_cap”.

TABLE 1
Control Loci Capture Probe and qPCR Information

| | | qPCR Assay & | | | | | Avg. |
|---|---|---|---|---|---|---|---|
| | | Control Locus Number: | | | | | of |
| Design Name | | 237 | 247 | 268 | 272 | Avg. | Avg's. |
| Berlin_basic | Sequence Species: | H | H | H | H | | |
| | Density (per bp): | 0.04 | 0.04 | 0.04 | 0.04 | | |
| | Copy Number: | 1 | 1 | 1 | 1 | | |
| | Measured | 356 | 102 | 385 | 373 | 304 | 208 |
| | Enrichment: | | | | | | |
| | Measured | 117 | 73 | 158 | 100 | 112 | |
| | Enrichment: | | | | | | |
| Berlin_test1 | Sequence Species: | H | H | H | H | | |
| | Density (per bp): | 0.50 | 0.50 | 0.50 | 0.50 | | |
| | Copy Number: | 1 | 1 | 1 | 1 | | |
| | Measured | 4532 | 1037 | 3625 | 2107 | 2825 | 1826 |
| | Enrichment: | | | | | | |
| | Measured | 1322 | 478 | 839 | 666 | 826 | |
| | Enrichment: | | | | | | |
| Berlin_test2 | Sequence Species: | H | H | H | H | | |
| | Density (per bp): | 0.50 | 0.50 | 0.50 | 0.50 | | |
| | Copy Number: | 5 | 5 | 5 | 5 | | |
| | Measured | 2346 | 498 | 2051 | 679 | 1394 | 1900 |
| | Enrichment: | | | | | | |
| | Measured | 3706 | 959 | 2898 | 2066 | 2407 | |
| | Enrichment: | | | | | | |
| Berlin_test3 | Sequence Species: | H | H | H | H | | |
| | Density (per bp): | 0.04 | 0.04 | 0.04 | 0.04 | | |
| | Copy Number: | 5 | 5 | 5 | 5 | | |
| | Measured | 1394 | 401 | 1195 | 880 | 968 | 966 |
| | Enrichment: | | | | | | |
| | Measured | 1883 | 383 | 1022 | 567 | 964 | |
| | Enrichment: | | | | | | |
| Berlin_test4 | Sequence Species: | H | H | H | H | | |
| | Density (per bp): | 0.04 | 0.04 | 0.04 | 0.04 | | |
| | Copy Number: | 1 | 1 | 1 | 1 | | |
| | Sequence Species: | M | M | M | M | | |
| | Density (per bp): | 0.04 | 0.04 | 0.04 | 0.04 | | |
| | Copy Number: | 1 | 1 | 1 | 1 | | |
| | Measured | 408 | 182 | 377 | 229 | 299 | 298 |
| | Enrichment: | | | | | | |
| | Measured | 487 | 63 | 500 | 140 | 298 | |
| | Enrichment: | | | | | | |
| Berlin_test7 | Sequence Species: | H | H | H | H | | |
| | Density (per bp): | 0.04 | 0.50 | 0.04 | 0.50 | | |
| | Copy Number: | 1 | 1 | 1 | 1 | | |
| | Measured | 510 | 769 | 550 | 2053 | 970 | 1035 |
| | Enrichment: | | | | | | |
| | Measured | 565 | 924 | 580 | 2326 | 1099 | |
| | Enrichment: | | | | | | |
| Berlin_test8 | Sequence Species: | H | H | H | H | | |
| | Density (per bp): | 0.50 | 0.04 | 0.50 | 0.04 | | |
| | Copy Number: | 1 | 1 | 1 | 1 | | |
| | Measured | 1296 | 65 | 995 | 109 | 616 | 1099 |
| | Enrichment: | | | | | | |
| | Measured | 3486 | 159 | 2488 | 191 | 1581 | |
| | Enrichment: | | | | | | |
| SickKids_basic | Sequence Species: | H | H | H | H | | |
| | Density (per bp): | 0.04 | 0.04 | 0.04 | 0.04 | | |
| | Copy Number: | 1 | 1 | 1 | 1 | | |
| | Measured | 71 | 47 | 187 | 107 | 103 | 135 |
| | Enrichment: | | | | | | |
| | Measured | 179 | 82 | 214 | 196 | 168 | |
| | Enrichment: | | | | | | |
| SickKids_test5 | Sequence Species: | M | M | M | M | | |
| | Density (per bp): | 0.04 | 0.04 | 0.04 | 0.04 | | |
| | Copy Number: | 1 | 1 | 1 | 1 | | |
| | Measured | 281 | 112 | 261 | 410 | 266 | 343 |
| | Enrichment: | | | | | | |
| | Measured | 429 | 169 | 584 | 496 | 419 | |
| | Enrichment: | | | | | | |
| SickKids_test6 | Sequence Species: | H | H | H | H | | |
| | Density (per bp): | 0.04 | 0.04 | 0.04 | 0.04 | | |
| | Copy Number: | 1 | 1 | 1 | 1 | | |
| | Sequence Species: | M | M | M | M | | |
| | Density (per bp): | 0.04 | 0.04 | 0.04 | 0.04 | | |
| | Copy Number: | 1 | 1 | 1 | 1 | | |
| | Measured | 436 | 167 | 474 | 779 | 464 | 418 |
| | Enrichment: | | | | | | |
| | Measured | 342 | 123 | 464 | 561 | 373 | |
| | Enrichment: | | | | | | |

H = Human;
M = Mouse

Human Genomic DNA (Promega Corp., Cat#G1471) and mouse genomic DNA (Novagen, Cat#69239-3) were obtained and utilized for testing for enrichment of sequences by the microarrays. Amplification primers were created for performing the qPCR of the control loci:

Assay 237

(SEQ ID NO:1)

| | |
|---|---|
| Forward Primer: | CGCATTCCTCATCCCAGTATG |

(SEQ ID NO:2)

| | |
|---|---|
| Reverse Primer: | AAAGGACTTGGTGCAGAGTTCAG |
| Assay 247 | |

(SEQ ID NO:3)

| | |
|---|---|
| Forward Primer: | CCCACCGCCTTCGACAT |

(SEQ ID NO:4)

| | |
|---|---|
| Reverse Primer: | CCTGCTTACTGTGGGCTCTTG |
| Assay 268 | |

(SEQ ID NO:5)

| | |
|---|---|
| Forward Primer: | CTCGCTTAACCAGACTCATCTACTGT |

(SEQ ID NO:6)

| | |
|---|---|
| Reverse Primer: | ACTTGGCTCAGCTGTATGAAGGT |
| Assay 272 | |

(SEQ ID NO:7)

| | |
|---|---|
| Forward Primer: | CAGCCCCAGCTCAGGTACAG |

(SEQ ID NO:8)

| | |
|---|---|
| Reverse Primer: | ATGATGCGAGTGCTGATGATG |

Sequence capture and qPCR were performed as described in the NimbleGen Arrays User's Guide for Sequence Capture Array Delivery (incorporated herein by reference in its entirety). Quantitative PCR was performed using the LightCycler 480 instrument (Roche) in 384-well format as defined by the manufacturer.

As exemplified in FIG. 1, the baseline design “Berlin_basic” (average probe density=0.04 capture control probes per base pair of target sequence, one copy of each different capture control probe on the array) shows an average enrichment (determined from 4 control capture loci and 2 replicate arrays) of 208-fold. Increasing the average probe density from 0.04 to 0.5 capture control probes per base pair of target sequence (design “Berlin_test1”), with no other changes, increased the average enrichment to 1826-fold. Increasing the copy number of each different capture control probe on the array to from 1 to 5 (design “Berlin_test3”), with no other changes, increased the average enrichment to 966-fold. Increasing the copy number of each different capture control probe on the array to from 1 to 5 while at the same time increasing the average probe density from 0.04 to 0.5 capture control probes per base pair of target sequence (design “Berlin_test2”) increased the average enrichment to 1900-fold. Design “Berlin_test4” demonstrates exemplary results of Sequence Capture of human DNA when the arrays comprised one set of control locus capture probes designed from human DNA (density 0.04, copy number 1) and one orthologous set of control locus capture probes designed from mouse DNA (density 0.04, copy number 1). The addition of the mouse control locus capture probes increased enrichment to 298-fold, which is approximately 44% greater than the baseline (design Berlin_basic). Increasing the average probe density from 0.04 to 0.5 capture control probes per base pair of target sequence for only 2 of the 4 loci (designs “Berlin_test7” and “Berlin_test8”), with no other changes, increased the average enrichment to 1035-fold and 1099-fold, respectively.

The design “SickKids_basic” comprised probes designed from mouse sequence to capture the targets but were created from human sequence to capture the control locus targets, producing an enrichment of 135-fold when capturing mouse DNA. Altering the control locus capture probes to coincide with the orthologous mouse sequences (“SickKids_test5”), with no other changes, produced an enrichment of 343-fold (154% greater than the baseline design (“SickKids_basic”). The design “SickKids_test6” combined the set of human probes from the design “SickKids_basic” and the set of orthologous mouse probes from the design “SickKids_test5” to produce an enrichment of 418-fold, which is 13% different than the enrichment from the simple addition of enrichments obtained by “SickKids_basic” and “SickKids_test5”.

Therefore, the use of the control loci for determining efficacy of sequence enrichment is demonstrated. Further, optimizing the parameters of the enrichment designs results in an increase in enrichment by Sequence Capture. For example, increasing the density of control locus capture probes per base pair of target sequence, increasing the copy number of control locus capture probes on the array, and/or increasing the sequence homology of the control locus capture probes to the species whose DNA is the target of the experiment has an influence on determining the efficacy of enrichment on a microarray assay.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method for determining enrichment of nucleic acid sequences from a hybridization assay comprising:
a) providing:
i) a nucleic acid sample comprising nucleic acid sequences,
ii) probes comprising the nucleic acid sequences as found on the nucleic acid sample,
b) hybridizing the nucleic acid sample with the probes thereby capturing the nucleic acid sequences, and
c) comparing the amount of nucleic acid sequences before hybridization to the amount of nucleic acid sequences captured by hybridization, thereby determining enrichment of nucleic acid sequences.

2. The method of claim 1, wherein said nucleic acid sequences are conserved nucleic acid sequences.

3. The method of claim 2, wherein said nucleic acid sequences further comprise target nucleic acid sequences.

4. The method of claim 1, where said hybridization assay is a microarray assay.

5. The method of claim 1, wherein said nucleic acid sample is a genomic nucleic acid sample.

6. The method of claim 5, wherein said genomic nucleic acid sample is a DNA sample.

7. The method of claim 1, wherein said comparing comprises performing polymerase chain reaction on the captured conserved nucleic acid sequences before hybridization and after hybridization.

8. The method of claim 7, wherein said polymerase chain reaction is quantitative polymerase chain reaction.

9. The method of claim 1, wherein said determining enrichment comprises determining fold enrichment between the amount of nucleic acid sequences captured prior to hybridization as compared to after hybridization.

10. The method of claim 1, wherein said enriching comprises removing non-hybridized nucleic acids by washing.

11. The method of claim 10, wherein said enriching further comprises eluting the hybridized and washed nucleic acids.

12. A method for determining enrichment of nucleic acid sequences from a microarray assay comprising:
a) providing a nucleic acid sample comprising nucleic acid sequences,
b) applying said sample to a substrate wherein said substrate comprises probes hybridizable to said nucleic acid sequences,
c) allowing hybridization capture to occur between the sample and the probes,
d) washing the hybridization capture,
e) eluting the captured nucleic acid sequences, and
d) comparing the amount of nucleic acid sequences before hybridization to the amount of nucleic acid sequences captured by hybridization, thereby determining enrichment of nucleic acid sequences from a microarray assay.

13. The method of claim 12, wherein said nucleic acid sequences are conserved nucleic acid sequences.

14. The method of claim 13, wherein said nucleic acid sequences further comprise target nucleic acid sequences.

15. The method of claim 12, wherein said nucleic acid sample is a genomic nucleic acid sample.

16. The method of claim 15, wherein said genomic nucleic acid sample is a DNA sample.

17. The method of claim 12, wherein said comparing comprises performing polymerase chain reaction on the captured conserved nucleic acid sequences before hybridization and after hybridization.

18. The method of claim 17, wherein said polymerase chain reaction is quantitative polymerase chain reaction.

19. The method of claim 12, wherein said determining enrichment comprises determining fold enrichment between the amount of nucleic acid sequences captured prior to hybridization as compared to after hybridization.

20. A kit for determining enrichment of nucleic acid sequences in a hybridization assay comprising a substrate, probes affixed to said substrate wherein said probes are homologous to one or more conserved nucleic acid sequences in a sample and primers homologous to said conserved nucleic acid sequences, wherein said primers are utilized to perform polymerase chain reaction of said conserved nucleic acid sequences for determining enrichment of nucleic acid sequences.

21. The kit of claim 20, further comprising reagents or solutions for performing one or more of hybridization, washing and elution.

22. The kit of claim 21, further comprising one or more of a polymerase, a ligase, a kinase, and a terminal transferase.
